(19) United States
(12) Patent Application Publication
(10) Pub. No.: US 2023/0098038 A1
(43) Pub. Date: Mar. 30, 2023

(54) Eyewear-based System and Method to Mechanically Increase Intraocular Pressure

(71) Applicant: West Virginia University, Morgantown, WV (US)

(72) Inventors: Abigail Osborne, Morgantown, WV (US); Emma Robertson, Lincoln University, PA (US); Ross Fontana, Winston-Salem, NC (US); Taylor Stump, Morgantown, WV (US); Hassan Alkhadrawi, Morgantown, WV (US); Jessica Allen, Gainesville, FL (US); Joel Palko, Morgantown, WV (US)

(21) Appl. No.: 17/936,384

(22) Filed: Sep. 29, 2022

Related U.S. Application Data
(60) Provisional application No. 63/249,660, filed on Sep. 29, 2021.

(51) Int. Cl. A61B 3/16; A61B 3/00; A61B 3/10

(52) U.S. Cl. CPC A61B 3/16; A61B 3/0075; A61B 3/102

(57) ABSTRACT

In one aspect, the present disclosure relates to an eyewear-based system and method comprised of an ocular compression device positioned around a user's head to compress the globes of a user's eyes using direct mechanical force to artificially elevate the user's intraocular pressure. An application of this system is to increase intraocular pressure in users with glaucoma or users who are at risk for glaucoma to assess novel biomarkers for glaucoma progression. The ocular compression device may be used in conjunction with an ocular imaging device, such as an optical coherence tomography angiography device, to characterize changes in the user's optic nerve head structure as a function of intraocular pressure. This abstract is intended as a scanning tool for purposes of searching in the particular art and is not intended to be limiting of the present disclosure.

### CROSS REFERENCE TO RELATED APPLICATIONS

This application claims the benefit of U.S. provisional application entitled “Eyewear-based System and Method to Mechanically Increase Intraocular Pressure” having Ser. No. 63/249,660, filed Sep. 29, 2021, which is hereby incorporated by reference in its entirety.

### FIELD OF THE INVENTION

This invention relates generally to systems and methods in the medical field of ophthalmology to artificially elevate the intraocular pressure of a user who may be at risk for glaucoma to study glaucoma progression. More specifically, this invention relates to an ocular compression device that is readily compatible with an ocular imaging device to characterize changes to a user's eye as a function of intraocular pressure.

### BACKGROUND OF THE INVENTION

Glaucoma is a leading cause of irreversible blindness. The etiology of glaucoma has yet to be elucidated. However, all types of glaucoma are associated with a neurodegeneration of the optic nerve head that cannot be regenerated [1]. Currently, the only modifiable risk factor in glaucoma within the eye is intraocular pressure (IOP). If left untreated, glaucoma can lead to visual field defects resulting in complete or partial vision loss. Furthermore, elevated IOP is neither necessary nor sufficient to induce glaucomatous damage, indicating that the interaction between IOP and variables such as ocular biomechanics and blood flow to the eye may contribute to glaucoma pathophysiology as well. Given that current diagnostic imaging in glaucoma is limited to measurements of the eye at a relatively static IOP, there is a current need for mechanisms that allow for dynamic imaging of the eye at controlled and varying IOP magnitudes. Exposing the eye to different environmental pressures and studying the results of these pressures on the hypothesized pathophysiologic mechanisms of glaucoma, such as biomechanics and ocular profusion, is vital to both risk stratifying glaucoma patients and understanding glaucoma progression. Novel diagnostic tools capable of characterizing the eye's response to a dynamically controlled IOP will help advance future diagnostic and treatment options of those suffering from glaucoma by revealing new biomarkers for glaucoma progression [2].

IOP is driven by the flow of aqueous humor fluid. Aqueous humor fluid is produced within the eye by the ciliary body and circulates to the anterior chamber with a flow rate of about 2 to 2.5 microliters per minute. This fluid is vital to stabilize the components of the eye and to supply the chambers of the eye with nutrition and to remove waste. Most aqueous fluid exits the eye through its conventional outflow system, which requires the fluid to flow to the surface of the eye via aqueous veins and eventually reabsorb into the orbital venous system. When this outflow system is intact, IOP typically ranges from 10 to 21 mm Hg. When the fluid does not drain properly, it accumulates in the anterior chamber causing ocular hypertension. The increase in IOP is a significant risk factor for the development of glaucomatous optic nerve neuropathy.

The eye is a fluid-filled structure with an average mass of about 8 grams and an average diameter of about 24 mm. The internal components of the eye are responsible for detecting light of different wavelengths as a first step in the visual pathway. The innermost layer of the posterior human eyes consists of the retina that detects lights through rods and cones, the major photosensitive cells of the retina.

The intermediate, uveal layer of the eye is vital to the circulation of the fluid in the eye due to the existence of the ciliary body and the pupil within that layer. Aqueous humor fluid is produced by the ciliary body epithelium of the eye at a rate of about 1 to 3 microliters per minute. Maintaining the same rate, it passes through the pupil to the anterior chamber. At the junction between the sclera and the cornea in the anterior chamber, the trabecular meshwork and Schlemm's canal function drains the aqueous humor out of the chamber to maintain IOP via the traditional outflow pathway [3]. The outermost layer of the eye has the sclera and the cornea.

The corneoscleral shell is a fibrous and collagenous coating that expands minimally and is the major load bearing structural of the eye. However, it has a geometric opening where the retinal ganglion cell axons converge to form the optic nerve and exit the eye. The optic nerve then travels with its approximate 1 million axons through the optic canal and to the optic chiasm. From here, the nerve, carrying all visual information from the retina, travels to the lateral geniculate nucleus to reach the occipital lobe, which functions as the imaging processing center. It is the inner most portion of the optic nerve as it exits the eye (i.e., the optic nerve head) that is damaged from IOP and other variables in glaucoma. The cascade of events starting at the optic nerve head that leads to retinal ganglion cell death in glaucoma remains unclear.

Optical Coherence Tomography (OCT) is a non-invasive diagnostic tool used to track glaucoma development and progression by measuring the thickness of the retinal nerve fiber layer (RNFL). As glaucoma worsens, the RNFL thins, and is a sensitive marker for glaucoma progression. OCT is also capable of measuring layers of the optic nerve head, such as the anterior portion of the lamina cribrosa, which is the collageneous support structure of the optic nerve head. It has been shown that the geometry and movement (i.e., strain) of the lamina cribrosa and its surrounding tissues with IOP changes has prognostic value in patients with glaucoma [4]. However, the inability to precisely control IOP at different levels during these dynamic OCT measurements limits their utilization in a clinical setting.

Optical Coherence Tomography Angiography (OCTA) is a non-invasive imaging method used to measure the optic nerve head, choroidal and retinal perfusion. OCTA can be used to help diagnose glaucoma, track its progression, and better understand its pathophysiology. OCTA can obtain multiple scans of the eye to analyze the flow of red blood cells and vascular change in various locations within the internal eye. It has been shown in some patients that a reduction in perfused vasculature to the optic nerve head and peripapillary choroid measured using OCTA is a risk factor for glaucoma. However, OCTA is currently limited to measurements during static IOP states only, limiting the diagnostic and research abilities of the device via incomplete dynamic vascular measurements around the optic nerve head.

The most common devices used to measure IOP are various types of tonometers. Ophthalmodynometers have also been traditionally used to elevate IOP by the application of force to the sclera to assess systematic vascular health and ocular perfusion. Ophthalmodynometer devices can have significant drawbacks in terms of application complexity, stability during testing, accuracy, repeatability, and limited data acquisition capability. Invasive and non-invasive methods have been used to elevate IOP by the application of force while testing for glaucoma. However, these methods are limited in their ability to be translated efficiently into the glaucoma clinic.

Major types of glaucoma are open-angle glaucoma, angle-closure glaucoma, and normal-tension glaucoma. In open angle glaucoma, it is believed that the trabecular meshwork loses its ability to allow aqueous humor to pass through it, resulting in IOP elevation and eventual glaucomatous damage. In primary angle-closure glaucoma, the pupil blockage pushes the iris closer to the trabecular meshwork which prevents the outflow of the aqueous humor. Furthermore, the lens moves forward toward the cornea, narrowing the angle between the iris and the cornea which impedes the fluid to properly circulate within the chambers of the eye. The greater the angle blockage, the greater the rise of IOP that may eventually damage the optic nerve. Those with normal-tension glaucoma have a normal IOP. However, the diagnosis of glaucoma is still detected by inspecting damage on the optic disc.

Detecting glaucoma is the first key to treatment and to avoid increased severity that could lead to blindness. If glaucoma is detected early, treatments may consist of medications or laser surgery to lower IOP and open the blockage to improve the circulation of aqueous humor. A variety of eye drops with mechanisms ranging from aqueous suppression to remodeling or stretching of the trabecular meshwork are used to lower IOP. Laser procedures, such as selective laser trabeculoplasty can be used to improve the conventional outflow of the eye. Surgery to remove trabecular meshwork or stent open and dilate Schlemms canal are currently used to also improve the conventional outflow pathway. Filtering surgeries, which create a new outflow path for aqueous using a variety of techniques and devices are used in more severe cases of glaucoma.

### SUMMARY OF THE INVENTION

The following specification, in at least one embodiment of the invention and broadly described herein, is an eyewear-based system and method comprised of an ocular compression device (OCD) positioned around a user's head to compress the globes of the user's eyes using direct mechanical force to artificially increase the user's IOP. One application of the ocular compression device (OCD) is to increase IOP in patients with glaucoma or parients who are at risk for glaucoma to assess novel biomarkers for glaucoma progression. The extraocular compression of the globes restricts the flow of aqueous humor through the veins and applies direct, controlled pressure to the globes, allowing for the IOP to increase as well as stay at the desired elevated IOP. The OCD is compatible with ocular imaging devices (OIDs), such as but not limited to an optical coherence tomography (OCT) or optical coherence tomography angiography (OCTA) devices, to characterize the change in the optic nerve head structure in addition to retinal and choroidal vascular networks that supply the user's eyes as a function of IOP. The device may also be used in conjunction with high frequency ultrasound imaging systems or anterior segment OCT to determine the mechanical properties of the cornea. The OCD eases the prognosis process when diagnosing the optic nerve cup and advances the process of opthamology by providing the ability to analyze common imaging metrics of both eyes simultaneously and efficienctly at controlled, elevated IOP levels. This method reduces the amount of variation in examining the user for early signs of glaucoma using an OID modality that results in a more efficient diagnosis and more effective understanding of the development of glaucoma in patients using the OCD. This device can also be used to see the effect of IOP on other ocular diseases including but not limited to keratoconus and corneal ectasias.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a photograph of an example ocular compression device positioned on the head of a user, in accordance with various embodiments of the present disclosure.

FIG. 2 is a photograph of an example ocular compression device illustrating select components, in accordance with the various embodiments of the present disclosure.

FIG. 3 is photograph of an example ocular compression device illustrating the longitudinal direction and transverse direction, in accordance with the various embodiments of the present disclosure.

FIG. 4 is a photograph of a partial view of an example ocular compression device focused on example spanner and pressure probe components, in accordance with the various embodiments of the present disclosure.

FIG. 5 is a photograph of an example ocular compression device positioned on the head of a user operating in conjunction with an example ocular imaging device, in accordance with the various embodiments of the present disclosure.

FIG. 6 is a photograph of an example ocular compression device positioned on the head of an alternate user operating in conjunction with an example ocular imaging device, in accordance with the various embodiments of the present disclosure.

FIG. 7 is an illustration identifying the upper globe and lower globe of a user's eye, in accordance with the various embodiments of the present disclosure.

FIG. 8 is a line drawing illustrating an isometric view of an example ocular compression device with two example pressure probes, in accordance with the various embodiments of the present disclosure.

FIG. 9 is a line drawing illustrating an isometric view of an example ocular compression device with one example pressure probe, in accordance with the various embodiments of the present disclosure.

FIG. 10 is a line drawing illustrating a partial front view of an example ocular compression device showing an example orientation of an example pressure probe, in accordance with the various embodiments of the present disclosure.

FIG. 11 is a line drawing illustrating an example goggle body frame, in accordance with the various embodiments of the present disclosure.

FIG. 12 is a line drawing illustrating an example spanner, in accordance with the various embodiments of the present disclosure.

FIG. 13 is an alternate line drawing illustrating an example spanner, in accordance with the various embodiments of the present disclosure.

FIG. 14 is a line drawing illustrating an example pressure probe, in accordance with the various embodiments of the present disclosure.

FIG. 15 is an alternate line drawing illustrating an example pressure probe, in accordance with the various embodiments of the present disclosure.

FIG. 16 is a photograph of an example eye rest, in accordance with the various embodiments of the present disclosure.

FIG. 17 is a line drawing illustrating an isometric view of an example ocular compression device with four example pressure probes, in accordance with the various embodiments of the present disclosure.

FIG. 18 is a line drawing illustrating a front view of an example ocular compression device with four example pressure probes, in accordance with the various embodiments of the present disclosure.

FIG. 19 illustrates a system comprising an ocular compression device, an ocular imaging device, a controller, and a computing device in accordance with the various embodiments of the present disclosure.

FIG. 20 is example intraocular pressure user measurement data as a function of pressure probe knob rotations, in accordance with the various embodiments of the present disclosure.

FIG. 21 is example intraocular pressure measurement data of users as a function of time, in accordance with the various embodiments of the present disclosure.

FIG. 22 is additional example intraocular pressure measurement data of users as a function of time, in accordance with the various embodiments of the present disclosure.

FIG. 23 is image data of a user's eye recorded by an optical coherence tomography angiography device without extraocular pressure being applied, in accordance with the various embodiments of the present disclosure.

FIG. 24 is image data of a user's eye recorded by an optical coherence tomography angiography device with extraocular pressure being applied from an example ocular compression device, in accordance with the various embodiments of the present disclosure.

### DETAILED DESCRIPTION OF THE INVENTION

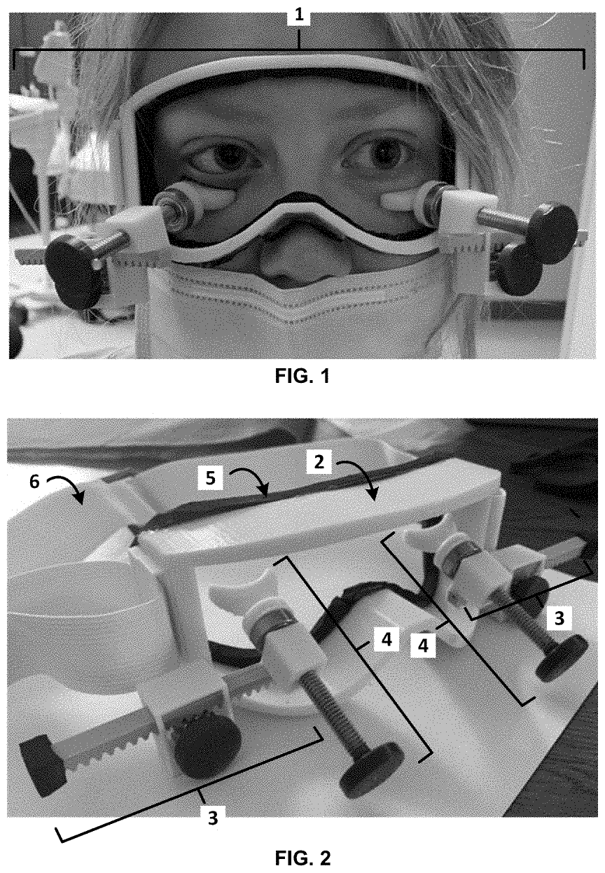

In one embodiment of the invention, an OCD 1 is comprised of a goggle body frame 2, at least one spanner 3, at least one pressure probe 4, a goggle body lining 5, and a headband 6 (see FIGS. 1-3).

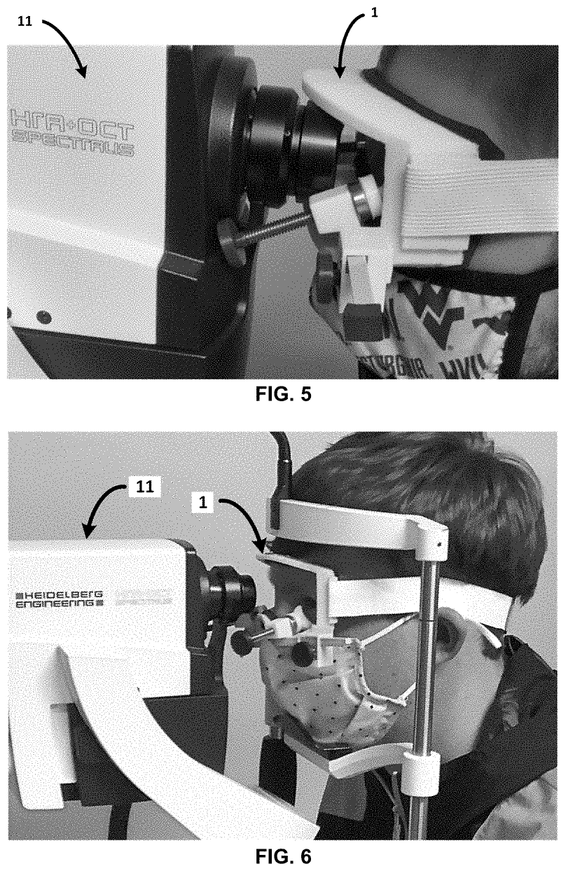

In a nonlimiting example, the goggle body frame 2 houses a plurality of spanners 3 connected to a plurality of pressure probes 4, respectively (see FIGS. 1-3, 8, 17, and 18). The fit of the OCD 1 allows for the plurality of pressure probes 4 to apply extraocular pressure (EOP) by compressing an upper globe 9 of a user's eye, a lower globe 10 of a user's eye, or a combination thereof using direct mechanical force to artificially increase IOP while the goggle body frame 2 rests on the user's face. The geometry, size, orientation, and field of motion of the OCD 1 components are designed to provide sufficient clearance for an OID 11, such as an OCTA device as a nonlimiting example, to collect imaging data of the user's eyes while artificially manipulating an increase in the IOP of the user's eyes (see FIGS. 5 through 7).

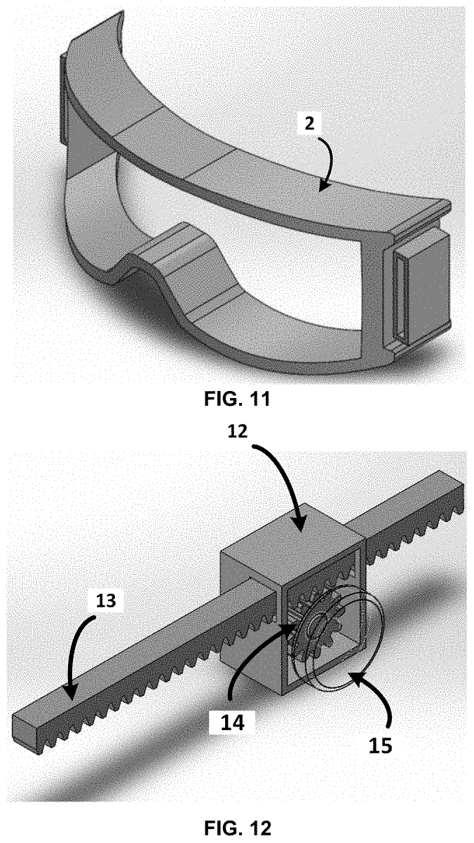

In a nonlimiting example, the goggle body frame 2 is comprised of a durable, semi-flexible material such as a polymer (see FIG. 11). The headband 6 is connected at each end to each side of the goggle body frame 2 using an elastic strap that contains an adjustable gear to increase or decrease the tension of the entire OCD 1 around the user's head to provide a comfortable fit for the user. The goggle body frame 2 is further comprised of a goggle body lining 5 where it contacts the user's face such as a foam lining, soft fabric, or other coating material like synthetic rubber designed to alleviate any discomfort the goggle body frame 2 may impart on the user's face, in addition to helping ease any tension the user may experience due to the tightening of the headband 6 around the user's head (see FIGS. 2, 5, and 6).

In one embodiment of the invention, the OCD 1 is comprised of two spanners 3 connected to two pressure probes 4, respectively, to compress the lower globe 10 of both eyes of the user (see FIGS. 1-3, 8).

In one embodiment of the invention, the OCD 1 is comprised of two spanners 3 connected to two pressure probes 4, respectively, to compress the upper globe 9 of both eyes of the user.

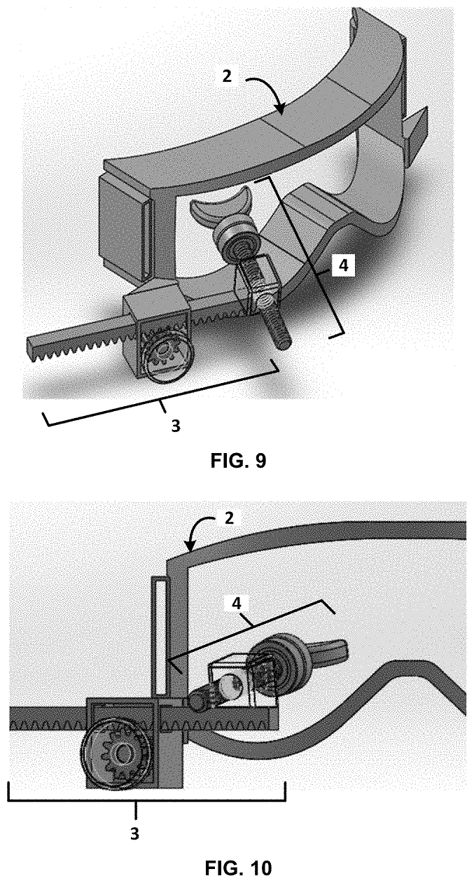

In one embodiment of the invention, the OCD 1 is comprised of one spanner 3 connected to one pressure probe 4, respectively, to compress the lower globe 10 of a single eye of the user (see FIG. 9).

In one embodiment of the invention, the OCD 1 is comprised of two spanners 3 connected to two pressure probes 4, respectively, to compress the upper globe 9 and lower globe 10 of a single eye of the user.

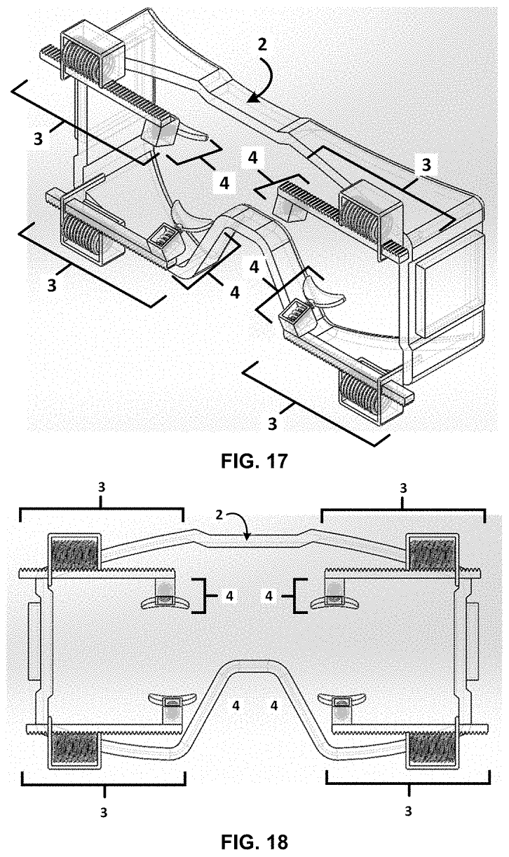

In one embodiment of the invention, the OCD 1 is comprised of four spanners 3 connected to four pressure probes 4, respectively, to compress the upper globe 9 and lower globe 10 of both eyes of the user (see FIGS. 17 and 18).

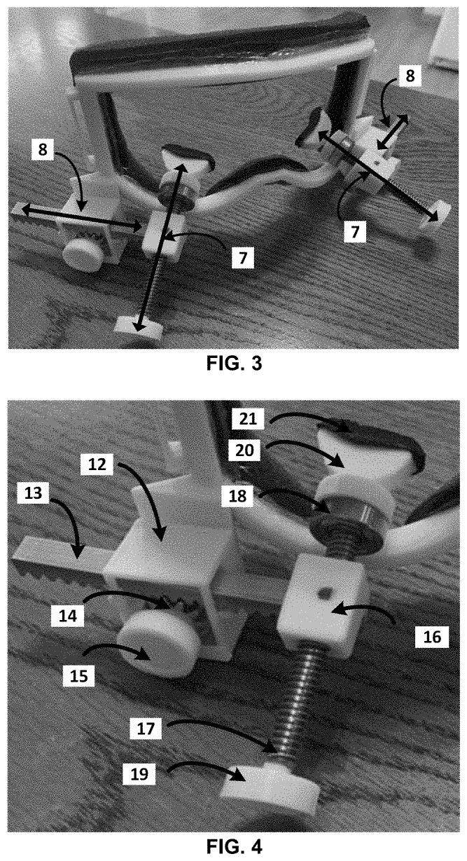

For spatial reference, the longitudinal direction 7 is defined as the direction approximately normal to a local ocular region of the user. The transverse direction 8 is defined as the direction approximately normal to the longitudinal direction 7 and approximately parallel to a line connecting the center of each of the user's eyes (see FIG. 3).

In a nonlimiting example, a plurality of spanners 3 connect to a plurality of pressure probes 4 wherein the plurality of spanners 3 is connected to a plurality of respective corners of the goggle body frame 2. The plurality of spanners 3 provide independent, continuous, transverse spatial manipulation of the plurality of pressure probes 4 with respect to the plurality of corners of the goggle body frame 2 to accomodate the user's unique facial anatomy (see FIGS. 1 through 3, 8 17, and 18).

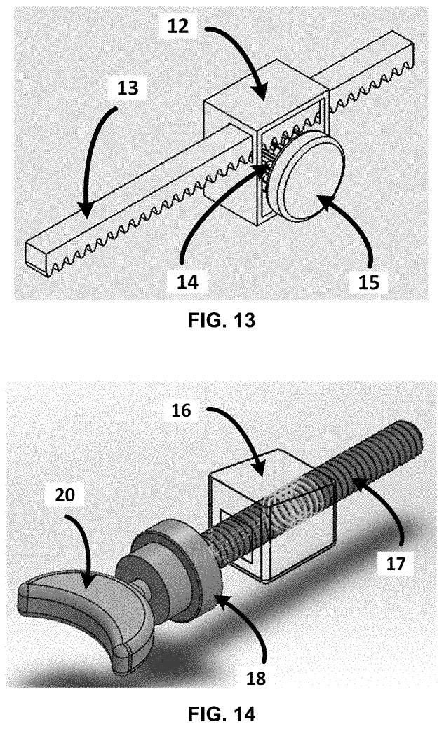

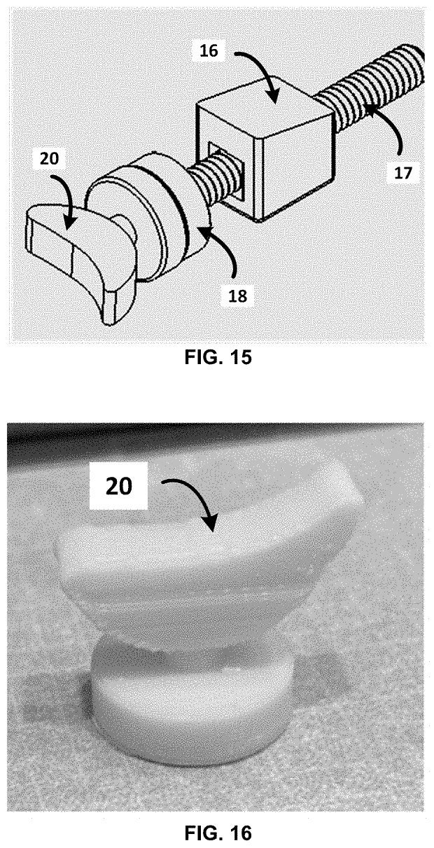

In one embodiment, a spanner 3 is an assembly of two main subcomponents: a spanner housing 12 and a spanner linear actuation subassembly comprised of a linear gear 13 and a circular gear 14. The spanner housing 12 supports the spanner linear actuation subassembly and is connected to a corner of the goggle body frame 2 (see FIGS. 1 through 4, 12 and 13). For manual adjustment of the spanner 3 in the transverse direction 8, a spanner knob 15 may be connected to the circular gear 14 and rotated by hand about the same axis as the circular gear 14 (see FIGS. 4, 12, and 13).

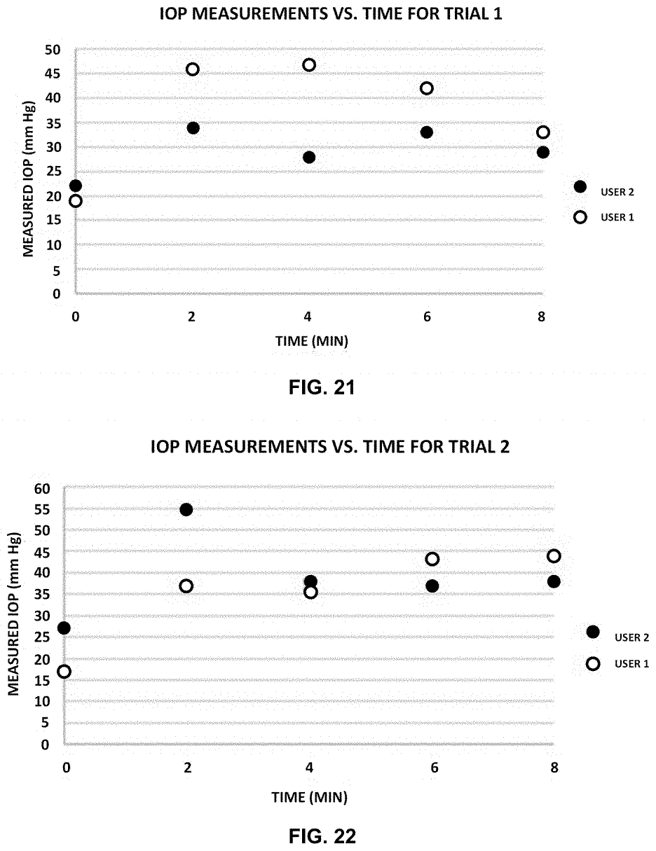

In a nonlimiting example, a plurality of pressure probes 4 connect to a plurality of respective spanners 3. The plurality of pressure probes 4 provide independent, continuous, longitudinal spatial manipulation to a plurality of eye rests 20 to apply EOP via compression against an upper globe 9, a lower globe 10, or a combination thereof (see FIGS. 1 through 4). An EOP is applied such that the plurality of eye rests 20 impart an increase in the IOP of the user's eyes. The lower limit of IOP is dependent and unique to the user's baseline IOP where no EOP is applied. A typical user's baseline IOP is within the approximate range of 10 to 21 mm Hg. An upper IOP of approximately 60 mm Hg was recorded as illustrated in FIG. 21 using the example OCD 1 shown in FIG. 1.

In one embodiment, the pressure probes 4 are an assembly of three main subcomponents: a pressure probe housing 16, an eye rest 20, and a pressure probe linear actuation subassembly comprised of a threaded rod 17 and a ball bearing 18. The pressure probe housing 16 supports the eye rest 20 and pressure probe linear actuation subassembly. The threaded rod 17 is supported near its proximal end within the pressure probe housing 16. The pressure probe housing 16 is internally threaded to mesh with the threaded rod 17. The proximal end of the pressure probe housing 16 is connected to the distal end of the spanner linear actuation subassembly. The eye rest 20 is connected to the distal end of the pressure probe linear actuation subassembly. The ball bearing 18 is connected to the eye rest 20 and threaded rod 17 such that the threaded rod 17 rotates to translate the eye rest 20 in the longitudinal direction 7 while keeping the orientation of the eye rest 20 approximately constant to conform to the local ocular region of the user's face when making contact. For manual adjustment of the pressure probe 4 in the longitudinal direction 7, a pressure probe knob 19 may be connected to the distal end of the threaded rod 17 and rotated by hand about the same axis as the threaded rod 17 (see FIGS. 1 through 4).

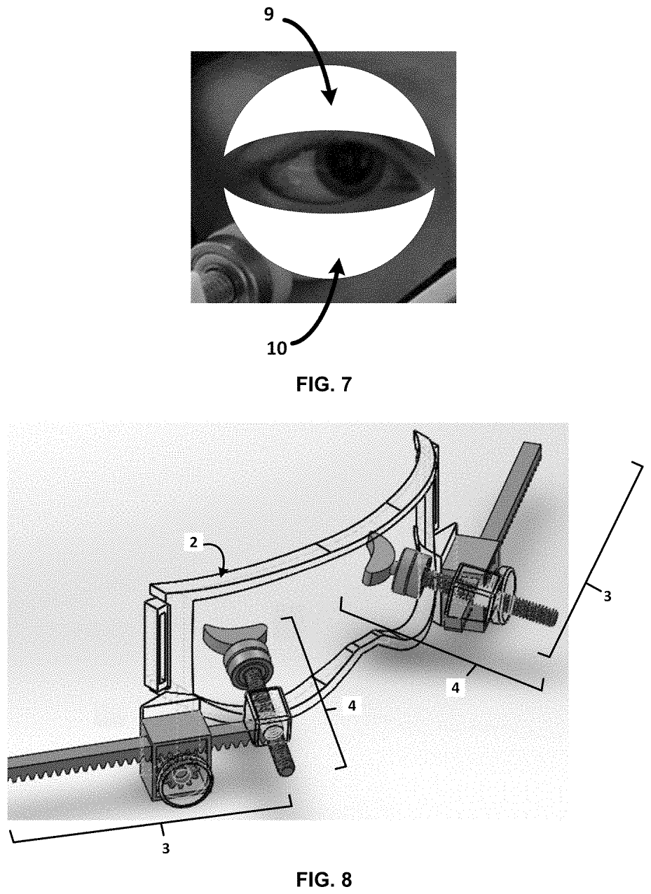

In a nonlimiting example, the eye rest 20 is in the shape of an asymmetric crescent to conform around the upper globe 9 or lower globe 10 of the user's eyes (see FIGS. 1 and 7). An eye rest lining 21 may be connected to the surface of the eye rest 20 that contacts the user's face comprised of similar materials as the goggle body lining 5 to alleviate any discomfort the eye rest 20 may impart on the user's face, in addition to helping ease any tension the user may experience due to the compression of the eye rest 20 against the user's face. The eye rest 20 provides a comfortable user experience by distributing a normal force to an upper globe 9 or lower globe 10 of the user's eyes across a large surface area. The asymmetric crescent shape of the eye rest 20 is preferred as it helps to create a better seal against each globe and increases the IOP imparted by the EOP by constricting more veins. This shape also helps accommodates the angle offset adjustment of the pressure probe 4 in the longitudional direction 7 to provide sufficient clearance for an OID 11 to operate in conjunction with the OCD 1 (see FIGS. 1 through 7 and 16).

In one embodiment, the spanner linear actuation subassembly, pressure probe linear actuation subassembly, or a combination thereof, may be manipulated by an electric motor or alternate means to provide linear actuation. A person having ordinary skill in the art would appreciate that there are many approaches to provide linear actuation that are applicable beyond the examples disclosed herein but are within the scope of this invention.

In one embodiment, a controller 22 is in electronic communication with the OCD 1 to manipulate the longitudinal position 7, transverse position 8, or a combination thereof, of at least one eye rest 20 to produce EOP and a resultant imparted IOP increase on the user's eyes.

In one embodiment, the controller 22 is also in electronic communication with an OID 11 to coordinate OID data acquisition as a function of eye rest 20 spatial manipulation by the controller 22.

In one embodiment, a system comprises a computing device 23 which is in electronic communication with the OCD 1, the OID 11, and a controller 22 such that, when instructed by an application 24, the computing device 23 commands the controller 22 to manipulate at least one eye rest 20 to impart EOP on at least one upper globe 9 or lower globe 10 of the user's eyes such that the IOP of at least one eye of the user increases. The application 24 further instructs the computing device 23 to command the OID 11 to perform data acquisition for at least a portion of the time while EOP is being applied. The application of EOP commanded by the computing device 23 may be time variant and may be in the form of a step function or ramping function to increase IOP over time, as nonlimiting examples.

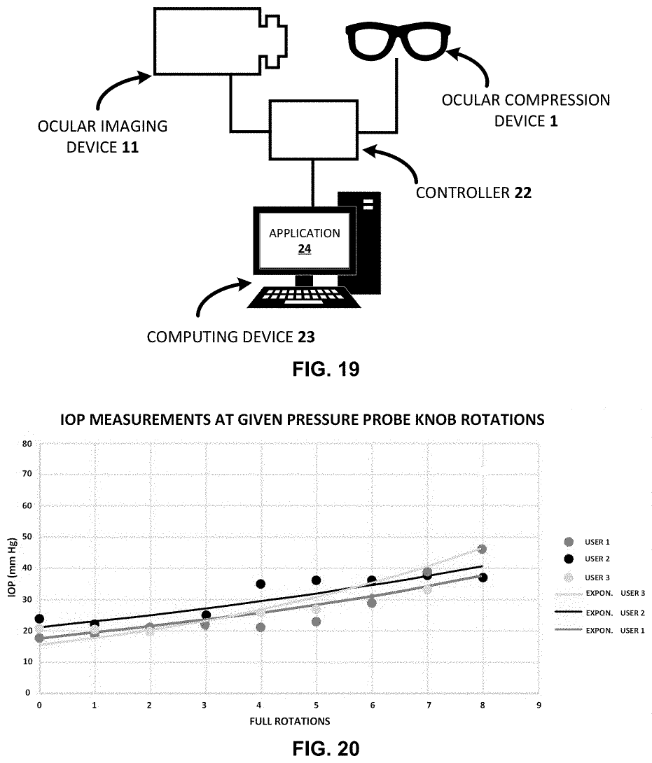

In a nonlimiting example, the application 24 instructs the computing device 23 to command the OID 11 to perform data acquisition for at least a portion of the time while EOP is being applied such that the eye rest 20 is receding and IOP decreases over time. These time varying, dynamic data acquisition aspects can help characterize stress-strain relationships within the eye and provide enhanced analysis of eye perfusion and eye re-perfrusion in a controlled manner. FIG. 19 illustrates the main components of this system.

This system can be used to characterize changes such as, but not limited to, ocular perfusion as a function of IOP, change in posterior globe strain as a function of IOP, and change in corneal strain as a function of IOP. These metrics can then be used as biomarkers for either diagnosing glaucoma or corneal disease such as Keratoconus/corneal ectasia or helping to understand a patient's risk of progressing with these diseases.

In one embodiment of the invention, the OCD 1 is designed to operate in conjunction with an OID 11 in an ophthalmologic setting for early glaucoma screenings by simulating an elevated IOP environment indicative of a user with glaucoma to examine the user's ocular vascular response. The OCD 1 is positioned around the user's head while resting the goggle body frame 2 on the user's face. The headband 6 is positioned around the user's head to provide a comfortable fit. Each spanner 3 is manipulated in the transverse direction 8 to align each respective pressure probe 4 such that each eye rest 20 is aligned with either an upper globe 9 or lower globe 10 of each eye. Each pressure probe 4 is then manipulated in the longitudinal direction 7 such that each eye rest 20 contacts the user's face and compresses the upper globe 9 or lower globe 10 of each respective eye using mechanical force. Once each spanner 3 and each pressure probe 4 are properly oriented, the user positions their face in view of an OID 11, such as but not limited to an OCTA device, for OID data acquisition of the user's eyes. Each pressure probe 4 is manipulated again to impart EOP on the user's eyes while OID data acquisition is in progress. As each pressure probe 4 is manipulated in the longitudinal direction 7, each eye rest 20 is pressed against the upper globe 9 or lower globe 10 of each eye to achieve a desired increase in IOP by constricting the flow of the aqueous homor through the veins. The upper The OCD 1 introduces EOP to alter the IOP of the trabecular meshwork and Schlemm's canal and by also compressing the tissue of the lower and upper eyes to block the flow of aqueous humor fluid through the circulating fluidic vessels of the eye. The EOP event may be a static event, a dynamic event, or a combination thereof.

In a nonlimiting example, an EOP event can be dynamic such that a decrease in IOP over time is recorded via OID data acquisition. This occurs after an elevated IOP has already been established and each eye rest 20 recedes from the upper globe 9 or lower globe 10 of each respective eye resulting in a reduced mechanical force over time, allowing IOP to decrease over time.

Manipulation of each spanner 3 and pressure probe 4 is respectively performed, as nonlimiting examples, manually by hand via spanner knob 15, pressure probe knob 19, automatically using an electric motor and controller 22, or a combination thereof. Manipulation of each spanner 3 and pressure probe 4 may also be performed independently, simultaneously, or a combination thereof.

In a nonlimiting example, the spanners 2 can be manually snap adjusted in the transverse direction 8 to align the pressure probes 4 with each eye. The pressure probes 4 then actuate such that each eye rest 20 applies equal compression to the upper globe 9 of the user's eyes, the lower globe 10 of the user's eyes, or a combination thereof, resulting in equal EOP applied to each of the user's eyes during an EOP event.

Example OCD 1 performance data were acquired from three users using the OCD 1 shown in FIG. 1 and the OID 11 shown in FIGS. 5 and 6. Those results are shown in Table 1 and FIGS. 20 through 24. A “full rotation” denotes a full rotation of the pressure probe knob 19 fixed to the end of the threaded rod 17 of the pressure probe linear actuation assembly. One full rotation resulted in translating each eye rest 20 approximately 1.27 mm closer to the lower globe 10 of each eye. The rotations were counted once after an initial point of contact was made with the lower globe 10 of each user's eye. Average IOP measurements of each set of eyes were recorded using a tonometer. The users reported their level of comfort from a range of 1 to 10, with 10 being the most comfortable. A paired t-distribution test was performed to determine if sufficient evidence existed to statistically determine whether the example OCD 1 could impart a significant increase in IOP. The calculated p-value of the t-distribution test was 0.0255, which compared to a standard alpha level of significance of 0.05, indicated there was a statistical difference in IOP from before the OCD 1 was used after eight full rotations, among the three users tested.

TABLE 1
User OCD Performance Data.

| Full | USER1 | | USER 2 | | USER 3 | |
|---|---|---|---|---|---|---|
| Rota- | | Comfort | | Comfort | | Comfort |
| tion | IOP | Scale | IOP | Scale | IOP | Scale |
| Count | (mm Hg) | (1-10) | (mm Hg) | (1-10) | (mm Hg) | (1-10) |
| 0 | 21 | 10 | 24 | 9 | 18 | 8 |
| 1 | 21 | 10 | 22 | 7 | 19 | 8 |
| 2 | 21 | 10 | 20 | 7 | 20 | 8 |
| 3 | 22 | 9 | 25 | 7 | 21 | 7 |
| 4 | 21 | 8 | 35 | 7 | 26 | 7 |
| 5 | 23 | 8 | 36 | 6 | 27 | 7 |
| 6 | 29 | 7 | 36 | 6 | 31 | 6 |
| 7 | 39 | 6 | 38 | 6 | 33 | 5 |
| 8 | 46 | 4 | 37 | 5 | — | 4 |
| 9 | — | — | 48 | 5 | — | — |

The example OCD 1 shown in FIG. 1 was further tested to determine the sensitivity of IOP measurements to time. FIGS. 21 and 22 illustrate measured IOP as a function of time for two users. These tests were performed with IOP measurements based on five full rotations of the threaded rods 17, recording IOP every two minutes for a total of eight minutes. Similar to the data collected in Table 1, full rotations were counted after the eye rests 20 made initial contact with the user's face. Overall, the data indicates there was a change in IOP from the start at time zero to eight minutes. It was hypothesized the fluctuations recorded here were due to user's holding their breath during the IOP measurement or possible inconsistency in measuring the IOP at each data sampling interval. A paired t-distribution test was also performed for these experiments to determine if sufficient evidence existed to statistically determine whether the example OCD 1 imparted a significant increase in IOP over time. The calculated p-value of the t-distribution test was 0.0211, which compared to a standard alpha level of significance of 0.05. This indicated there was a statistical difference in IOP from before the pressure probes 4 of the example OCD 1 were activated to up to eight minutes after the test was initiated.

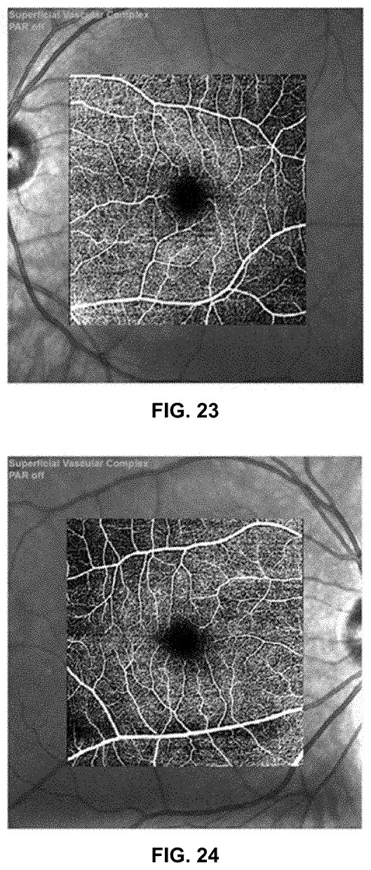

OID data was collected to further illustrate the performance of the example OCD 1 shown in FIG. 1. FIG. 23 shows OID image data collected from an OCTA device of a user's eye where EOP was not being applied, or a user's baseline IOP conditions. FIG. 24 shows OID image data collected from an OCTA device of a user's eye where EOP was applied by the example OCD 1 shown in FIG. 1. The imaging stability and similarity with and without the device, as shown in FIG. 23 and FIG. 24, are critical to image evaluation as IOP is changed and demonstrate the ability of the device to integrate into commonly used clinical imaging systems.

### REFERENCES

1. Jacobs, D. (2020, Aug. 4) Open-angle Glaucoma: Epidemiology, Clinical Presentation, and Diagnosis. Available from: https://www.uptodate.com/contents/open-angle-glaucoma-epidemiology-clinical-presentation-and-diagnosis
2. Kent, C. (2011, Jun. 13). IOP: Managing the Fluctuating Factor. Available from: https://wwv.reviewofophthalmology.com/article/iop-managing-the-fluctuation-factor
3. Sunderland D K, Sapra A. Physiology, Aqueous Humor Circulation. [Updated 2022 Jan. 8]. In: StatPearls [Internet]. Treasure Island (FL): StatPearls Publishing; 2022 January-. Available from: https://www.ncbi.nlm.nih.gov/books/NBK553209/
4. Chuangsuwanich T, Tun T A, Braeu F A, Wang X, Chin Z Y, Panda S K, Buist M, Strouthidis N, Perera S, Nongpiur M, Aung T, Girard M J, Differing Associations between Optic Nerve Head Strains and Visual Field Loss in Normal- and High-Tension Glaucoma Subjects, Ophthalmology (2022), doi: https://doi/org/10.1016/j.ophtha.2022.08.007

What is claimed is:

1. An ocular compression device for wearing on the face of a user, comprising:
a goggle body frame surrounding the eyes of the user wherein the goggle body frame has a transverse direction and a longitudinal direction;
at least one spanner connected to the goggle body frame;
at least one pressure probe connected to the at least one spanner;
wherein the at least one spanner is adjustable in the transverse direction to align the at least one pressure probe with at least one eye of the user; and
wherein the at least one pressure probe is adjustable in the longitudinal direction to contact the face of the user and apply extraocular pressure using mechanical force to elevate the intraocular pressure of at least one eye of the user.

2. The device of claim 1, wherein the goggle body frame is secured to the head of the user with a headband to provide fit for the head of the user.

3. The device of claim 1, wherein the goggle body frame is connected to a goggle body lining made of a soft material in contact with the face of the user to alleviate discomfort.

4. The device of claim 1, wherein the at least one pressure probe is adjacent to at least one lower globe of an eye or one upper globe of an eye.

5. The device of claim 1, wherein the ocular compression device is comprised of two spanners and two pressure probes such that each pressure probe is adjacent to the lower globe of each eye of the user.

6. The device of claim 1, wherein the ocular compression device is comprised of two spanners and two pressure probes such that each pressure probe is adjacent to the upper globe of each eye of the user.

7. The device of claim 1, wherein the ocular compression device is comprised of two spanners and two pressure probes such that each pressure probe is adjacent to the upper and lower globe of a single eye of the user.

8. The device of claim 1, wherein in the ocular compression device is comprised of four spanners and four pressure probes such that each pressure probe is adjacent to the upper and lower globe of each eye of the user.

9. The device of claim 1, wherein the spanner is comprised of a spanner housing and a spanner linear actuation subassembly.

10. The device of claim 1, wherein the pressure probe is comprised of a pressure probe housing, a pressure probe linear actuation subassembly, and an eye rest.

11. The device of claim 10, wherein the the eye rest is in the shape of an asymmetric crescent.

12. The device of claim 9, wherein the spanner linear actuation subassembly is manipulated manually by hand or automatically via an electric motor.

13. The device of claim 10, wherein the pressure probe linear actuation subassembly is manipulated manually by hand or automatically via an electric motor.

14. The device of claim 1, wherein the extraocular pressure is dynamic, static, or a combination thereof.

15. The device of claim 1, wherein the extraocular pressure imparts an intraocular pressure from about 5 mm Hg to about 60 mm Hg.

16. The device of claim 1, wherein the ocular compression device is used to screen the user for ocular hypertension, glaucoma, or a combination thereof.

17. The device of claim 1,
wherein the goggle body frame is secured to the head of the user with a headband to provide fit for the head of the user;
wherein the goggle body frame is connected to a goggle body lining made of a soft material in contact with the face of the user to alleviate discomfort;
wherein the ocular compression device is comprised of two spanners and two pressure probes such that each pressure probe is adjacent to the lower globe of each eye of the user;
wherein the spanner is comprised of a spanner housing and a spanner linear actuation subassembly;
wherein the pressure probe is comprised of a pressure probe housing, a pressure probe linear actuation subassembly, and an eye rest;
wherein the eye rest is in the shape of an axymmetric crescent;
wherein the spanner linear actuation subassembly is manipulated manually by hand or automatically via an electric motor;
wherein the spanner linear actuation subassembly is manipulated manually by hand or automatically via an electric motor;
wherein the extraocular pressure is dynamic, static, or a combination thereof.

18. A method to elevate the intraocular pressure of at least one eye of a user, comprising:
fitting an ocular compression device on the face of a user;
manipulating at least one spanner connected to the ocular compression device in the transverse direction to align at least one pressure probe with at least one eye of the user;
manipulating the at least one pressure probe connected to the at least one spanner in the longitudinal direction to contact the face of the user to apply extraocular pressure using mechanical force to elevate the intraocular pressure of at least one eye of the user.

19. The method of claim 18, wherein the application of extraocular pressure is used to screen the user for ocular hypertension, glaucoma, or a combination thereof.

20. The method of claim 18, wherein the extraocular pressure is dynamic, static, or a combination thereof.

21. A system for acquiring ocular data from a user, comprising:
an ocular compression device;
an ocular imaging device;
a computing device;
a controller,
wherein the computing device is in electronic communication with the controller which is in electronic communication with the ocular compression device;
wherein the computing device commands the controller to manipulate the ocular compression device to apply extraocular pressure by mechanical force to elevate the intraocular pressure of at least one eye of the user; and
wherein the computing device commands the controller to manipulate the ocular imaging device to record ocular data for the at least one eye of the user for at least a portion of the time the mechanical force is applied to the at least one eye.

22. The system of claim 21, wherein the ocular imaging device performs optical coherence tomography, optical coherence tomography angiography, or a combination thereof.

23. The system of claim 21, wherein the system is used to screen the user for ocular hypertension, glaucoma, or a combination thereof.

24. The system of claim 21, wherein the extraocular pressure is dynamic, static, or a combination thereof.
